(19) United States
(12) Patent Application Publication
(10) Pub. No.: US 2022/0226163 A1
(43) Pub. Date: Jul. 21, 2022

(54) COMPRESSION GARMENT

(71) Applicant: Tiffany Faith Haensel, Cochrane (CA)

(72) Inventor: Tiffany Faith Haensel, Cochrane (CA)

(21) Appl. No.: 17/150,414

(22) Filed: Jan. 15, 2021

(51) Int. Cl. A61F 13/08; A61F 13/10; A41D 19/00; A41D 31/10; A41D 31/12

(52) U.S. Cl. CPC A61F 13/08; A61F 13/104; A41D 31/125; A41D 31/10; A41D 19/0013

(57) ABSTRACT

A compression garment that is operable to provide a compression force to a portion of the body of the wearer and wherein the outer surface thereof is configured to be washed and sanitized. The compression garment of the present invention if further configured to provide absorption of any moisture present adjacent the skin of the wearer during wearing of the compression garment. The compression garment includes an outer layer and an inner layer. The outer layer is manufactured from an expandable, waterproof and solvent resistant material. An inner layer is adjacent the outer layer and is manufactured from an absorbent material. A plurality of expansion joints are present in the inner layer so as to inhibit damage to the inner layer during expansion of the outer layer. The compression garment is washable so as to be properly cleaned and sanitized.

### FIELD OF THE INVENTION

The present invention relates generally to compression garments, more specifically but not by way of limitation, a compression garment configured to provide compression force to a limb or extremity of a human wherein the garment of the present invention is waterproof, breathable and further provides absorption of sweat and moisture so as to facilitate the wearer of the present invention to don the garment for extended periods of time and further be able to properly sanitize the outer surface thereof.

### BACKGROUND

Millions of people suffer from various medical conditions that cause swelling in their arms, legs, hands or feet. Swelling can result from various conditions such as but not limited to arthritis or phlebitis. Extremity swelling can further be a post-surgical side effect especially if during the surgery either some or all of the lymph nodes were removed. It has been common practice for years to utilize compression to treat swelling of the extremities. The compression force applied by conventional compression gloves, stockings and the like are configured to apply a pressure force that enables lymphatic fluid to flow properly so as to reduce the swelling and further enable a person to perform regular activities.

One issue with conventional compression garments that can be worn for an extended period of time wherein the garment is potentially exposed to moisture or further where the wearer needs to execute sanitizing of the outer surface. Most conventional compression garments are manufactured from a single layer of material that is expandable so as to be donned on an extremity and apply a force thereto. These garments however cannot be properly sanitized and in the case of compression gloves, wearers thereof may not be permitted to wear them in certain areas. Furthermore, conventional compression garments are not waterproof and as such wearers thereof cannot be exposed to wet conditions for lengths of time without having to remove in order to inhibit damage to the skin. Additionally, engagement in activity that produces sweating creates a discomfort for the wearer of a conventional compression garment as some of the moisture is essentially trapped between the compression garment and the skin.

It is intended within the scope of the present invention to provide a compression garment manufactured from a material that is waterproof and further includes an outer layer that is solvent resistant and can be properly sanitized. Furthermore, the present invention includes an inner layer configured to expand with the outer layer and provide moisture absorption.

### SUMMARY OF THE INVENTION

It is the object of the present invention to provide a compression garment that is configured to be worn on an extremity or limb of a human and apply a compression force thereto wherein the compression garment of the present invention includes an outer layer and an inner layer.

Another object of the present invention is to provide a compression garment configured so as to provide the ability for the outer surface to be cleaned and/or sanitized wherein the outer layer is manufactured from an expandable material and is configured to apply the compression force ensuing the garment of the present invention being donned by a user.

A further object of the present invention is to provide a compression garment that is configured to be worn on an extremity or limb of a human and apply a compression force thereto wherein the compression garment of the present invention has an inner layer that is configured to provide absorption of moisture.

Still another object of the present invention is to provide a compression garment configured so as to provide the ability for the outer surface to be cleaned and/or sanitized wherein the inner layer further includes a plurality of expansion joints so as to be movable in conjunction with the outer layer.

An additional object of the present invention is to provide a compression garment that is configured to be worn on an extremity or limb of a human and apply a compression force thereto wherein the ends of the garment further include a sealing member that is configured to inhibit penetration of fluid between the garment and the skin of the user.

Yet a further object of the present invention is to provide a compression garment configured so as to provide the ability for the outer surface to be cleaned and/or sanitized wherein the outer layer is further solvent resistant in order to facilitate sanitation thereof utilizing a solvent such as but not limited to isopropyl alcohol.

Another object of the present invention is to provide a compression garment that is configured to be worn on an extremity or limb of a human and apply a compression force thereto wherein in a preferred embodiment of the present invention the garment is configured to apply a compression force between fifteen mm/Hg and fifty mm/Hg.

An alternate object of the present invention is to provide a compression garment configured so as to provide the ability for the outer surface to be cleaned and/or sanitized that includes embodiments such as but not limited to stockings, sleeves and gloves.

Still a further object of the present invention is to provide a compression garment that is configured to be worn on an extremity or limb of a human and apply a compression force thereto wherein it is further contemplated within the scope of the present invention that the outer surface can be impregnated with an antimicrobial compound.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is an exemplary three-quarter finger glove of the present invention; and

FIG. 2 is an exemplary full finger glove of the present invention; and

FIG. 3 is an exemplary stocking of the present invention; and

FIG. 4 is a cross-section of the material of the present invention.

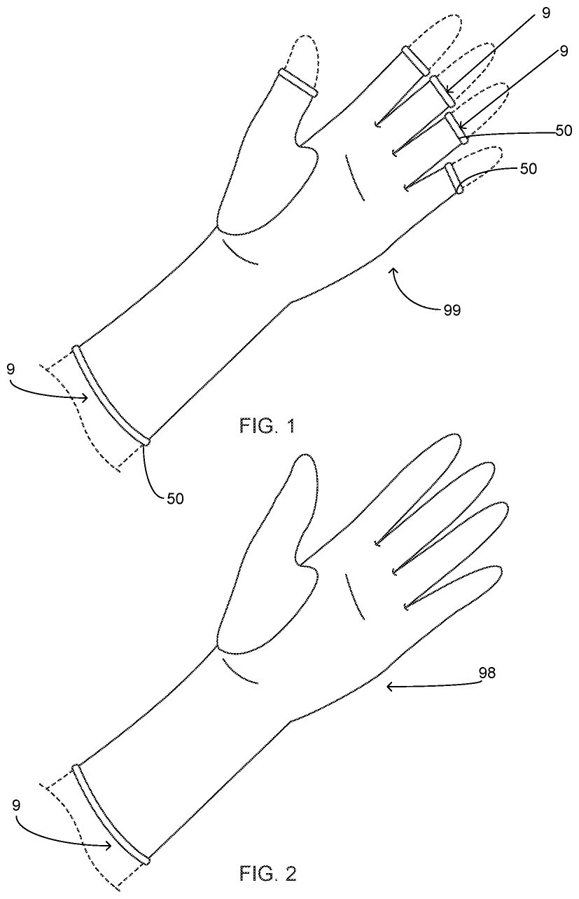

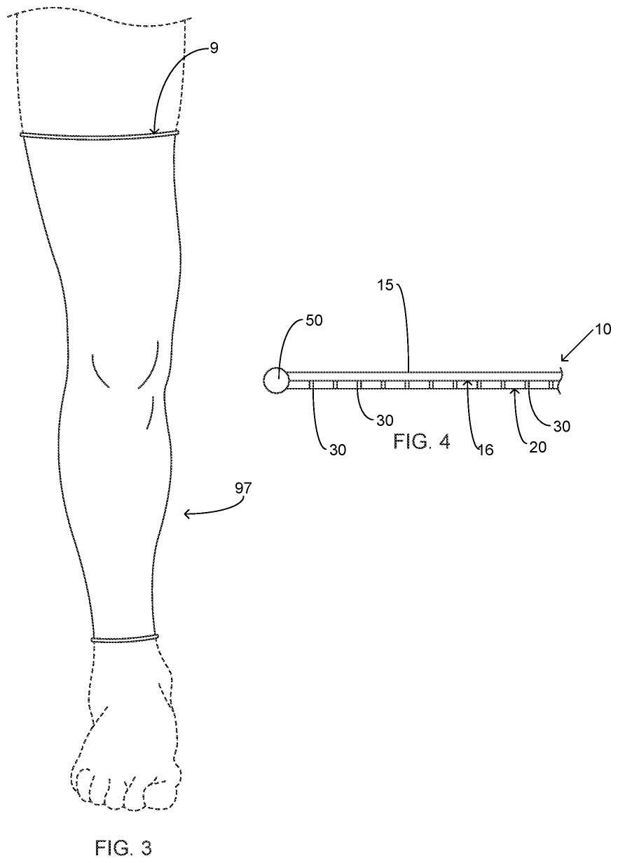

### DETAILED DESCRIPTION

References now to the drawings submitted herewith, wherein various elements depicted therein are not necessarily drawn to scale and wherein through the views and figures like elements are referenced with identical reference numerals, there is illustrated a compression garment 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Now referring to the Figures submitted herewith, the compression garment 100 includes a body 10 wherein the body 10 is comprised of an outer layer 15 and an inner layer 20. The outer layer 15 and inner layer 20 are adjacent each other and are bonded utilizing suitable durable techniques. The body 10 is formed into various garments such as but not limited to gloves and stockings. It should be understood within the scope of the present invention that while the compression garment 100 is illustrated herein as being formed in two glove embodiments 99, 98 and a leg stocking 97 that the compression garment 100 could be provided in alternate styles and/or sizes. The outer layer 15 of the body 10 is manufactured from a suitable expandable material that provides the necessary properties required for the present invention. The outer layer 15 of the present invention is expandable so as to facilitate application to a limb or extremity and subsequently provide a compression force thereto. The outer layer 15 is further configured to be waterproof and resistant to solvent in order to allow proper cleansing and/or sanitizing thereof without removal of the compression garment 100 from a wearer. It should be understood within the scope of the present invention that the outer layer 15 could be manufactured from alternate materials in order to provide the desired functionality as described herein.

The outer layer 15 is bonded to the inner layer 20. It should be understood within the scope of the present invention that the inner layer 20 and outer layer 15 are manufactured of the same size and present equally on the compression garment 100. The inner layer 20 is configured to be adjacent the wearer ensuing donning of the compression garment 100. The inner layer 20 is operable to absorb any moisture or sweat that may be present adjacent thereto and maintain that moisture away from the skin of the wearer. The inner layer 20 in a preferred embodiment of the present invention is manufactured from an absorbent polymer. It should be understood within the scope of the present invention that the inner layer 20 could be manufactured from various alternate absorbent materials. Furthermore, it is contemplated within the scope of the present invention that the inner layer 20 could be provided in alternate thicknesses.

As the present invention must inherently expand to accept a limb or extremity and then compress thereagainst, the inner layer 20 is configured to adapt to this movement that is provided by the outer layer 15. The inner layer 20 includes a plurality of expansion joints 30 wherein the expansion joints 30 are operable to allow the inner layer 20 to expand and contract the same amount as the outer layer 15 while inhibiting damage to the inner layer 20. Absorbent materials are not intrinsically expandable and once torn will lose their absorbency effectiveness. The expansion joints 30 facilitate the ability for the inner layer 20 to move in conjunction with the outer layer 15 and provide protection from damage thereto. In a preferred embodiment of the present invention, the expansion joints 30 are manufactured from the same material as the outer layer 15 wherein the expansion joints 30 are contiguously formed with the bottom surface 16 of the outer layer 15 and extend downward therefrom. While an embodiment of the expansion joints 30 are described herein, it should be understood within the scope of the present invention that the expansion joints 30 could be manufactured from alternate materials and with alternate techniques and still achieve the desired objective discussed herein. Furthermore, it should be understood within the scope of the present invention that there could be various quantities of expansion joints 30 deployed per linear inch of the compression garment 100 to ensure achievement of the desired objectives discussed herein.

A sealing member 50 is present at the ends 9 of the compression garment 100. The sealing member 50 is present so as to inhibit entry of fluids intermediate the compression garment 100 and the skin of the user during the wearing of the compression garment 100. The sealing member 50 is manufactured from a resilient material such as but not limited to silicone rubber. The sealing member 50 is provided in a bead shape and is secured to the ends 9 utilizing suitable durable techniques. As is illustrated in the embodiments herein, the sealing member 50 is present on the fingers of the three-quarter finger glove 99 as well and the cuff. For the leg stocking 97, the sealing member 50 is present at the distal ends thereof. It should be understood within the scope of the present invention that the sealing member 50 could be manufactured from alternate materials and be provided in alternate shapes and sizes in order to achieve the desired objective of inhibit water or fluid from propagating past the ends 9.

While not particularly illustrated herein, it is contemplated within the scope of the present invention that the outer layer 15 could be impregnated with an antimicrobial compound. Furthermore, it is additionally contemplated that the compression garment 100 can be removed, clean and re-used. Additionally, it is within the scope of the present invention that the inner layer 20 could be releasably secured to the bottom surface 16 of the outer layer 15 so as to facilitate the replacement thereof so a user can re-use the compression garment 100. It should also be understood that the compression garment 100 could be provided in various different sizes in order to accommodate wearers having different body and limb sizes.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding description is, therefore, not intended to be limited to the specific forms set forth herein, but on the contrary, it is intended to cover such alternatives, modifications, and equivalents, as can be reasonably included within the spirit and scope of the invention.

What is claimed is:

1. A compression garment configured to be donned by a wearer and apply a compression force wherein the compression garment comprises:
a body, said body having an outer layer and an inner layer, said outer layer and said inner layer being adjacent, said outer layer being manufactured from an expandable material, said outer layer configured to be expandable for donning of the compression garment and subsequently apply a compression force to a portion of a body of the wearer, said outer layer being waterproof, said inner layer configured to be adjacent the wearer ensuing donning of the compression garment, said inner layer being manufactured from an absorbent material, said inner layer being of equal size as the outer layer; and
a plurality of expansion joints, said plurality of expansion joints being distributed through said inner layer, said plurality of expansion joints being operable to inhibit damage to said inner layer during expansion of the compression garment.

2. The compression garment as recited in claim 1, wherein the plurality of expansion joints are integrally formed with a bottom surface of the outer layer, said plurality of expansion joints extending downward from said bottom surface of said outer layer.

3. The compression garment as recited in claim 2, wherein said body further includes at least one sealing member, said sealing member present at an end of the compression garment, said sealing member configured to inhibit water from propagating therepast.

4. The compression garment as recited in claim 3, wherein the compression garment is manufactured in an embodiment selected from a one of a following type: a leg stocking, a sleeve, a three-quarter finger glove or a full finger glove.

5. The compression garment as recited in claim 4, wherein the outer layer is resistant to solvents.

6. The compression garment as recited in claim 5, wherein compression force applied by the compression garment ranges from 15 to 50 mm/Hg.

7. The compression garment as recited in claim 1, wherein the inner layer is releasably secured to the outer layer.

8. The compression garment as recited in claim 7, wherein the outer layer is configured to be washable so as to provide cleaning thereof.

9. A compression garment that is configured to be cleaned and sanitized in order to facilitate a prolonged wearing thereof wherein the compression garment comprises:
an outer layer, said outer layer being manufactured from an expandable material, said outer layer further being manufactured from a waterproof material, said outer layer having a bottom surface, said outer layer configured to provide a compression force to a portion of a body of a user;
an inner layer, said inner layer being adjacent to said bottom surface of said outer layer, said inner layer being adjacent a user's skin ensuing donning of the compression garment, said inner layer being manufactured from an absorbent material, said inner layer being equivalent in size to said outer layer; and
a plurality of expansion joints, said plurality of expansion joints being present within said inner layer, said plurality of expansion joints being manufactured from an expandable material, said plurality of expansion joints configured to inhibit damage to said inner layer during expansion of the compression garment.

10. The compression garment as recited in claim 9, wherein the plurality of expansion joints are integrally formed with said bottom surface of said outer layer, said plurality of expansion joints extending downward from said bottom surface of said outer layer.

11. The compression garment as recited in claim 10, wherein said the compression garment further includes at least one sealing member, said sealing member present at an end of the compression garment, said sealing member configured to inhibit water from propagating therepast.

12. The compression garment as recited in claim 11, wherein the compression garment is selected from a group of one of the following garments: a leg stocking, a sleeve, a three-quarter finger glove or a full finger glove.

13. The compression garment as recited in claim 12, wherein said inner layer is manufactured from an absorbent polymer.

14. The compression garment as recited in claim 13, wherein compression force applied by the compression garment ranges from 15 to 50 mm/Hg.
